(19) United States
(12) Patent Application Publication
(10) Pub. No.: US 2024/0325283 A1
(43) Pub. Date: Oct. 3, 2024

(54) ARTIFICIAL NAIL COMPOSITION CONTAINING URETHANE POLYMER PARTICLES

(71) Applicant: SHOFU INC., Kyoto (JP)

(72) Inventors: Kazuki YAMAMOTO, Kyoto (JP); Shun SHIMOSOYAMA, Kyoto (JP)

(21) Appl. No.: 18/618,954

(22) Filed: Mar. 27, 2024

(30) Foreign Application Priority Data
Mar. 29, 2023 (JP) 2023-052706

(51) Int. Cl. A61K 8/87; A45D 31/00; A61K 8/81; A61Q 3/02

(52) U.S. Cl. CPC A61K 8/87; A45D 31/00; A61K 8/817; A61Q 3/02

(57) ABSTRACT

It is an object of the present invention to provide an artificial nail composition having a matte texture with reduced gloss and having high flexibility. The artificial nail composition of the present invention contains a component (A): a polymerizable compound, a component (B): urethane polymer particles, and a component (C): a polymerization initiator.

### FIELD OF INVENTION

The present invention relates to an artificial nail composition having a matte texture with reduced gloss and having high flexibility.

### BACKGROUND

Among artificial nails such as nail polishes, gel nails, and acrylic nails, gel nails are widely used from the viewpoint of good operability, long-term lasting properties on a nail or an artificial nail, and low odor. As one of gel nail designs, a gel nail having a matte texture with reduced gloss (no sheen) is required, and as one of methods for realizing such a gel nail having a matte texture, there is a method of adding a filler to a photocurable artificial nail composition to form irregularities on a surface of a cured film, and causing irregular reflection of light to reduce gloss.

As an artificial nail composition in which gloss is reduced by adding a filler as described above, an artificial nail composition exemplified in Japanese Patent No. 7014970 is known.

### SUMMARY OF INVENTION

However, since the artificial nail composition of Japanese Patent No. 7014970 contains (meth)acrylic polymer particles and has low flexibility after curing, there is a problem that the artificial nail composition cannot follow the movement of a nail or an artificial nail and is easily peeled off. As described above, the conventional artificial nail composition has difficulty in imparting high flexibility while having a matte texture with reduced gloss.

The present invention has been made in view of the above circumstances, and an object of the present invention is to provide an artificial nail composition having a matte texture with reduced gloss and having high flexibility.

The solution of the above problem is achieved by the following present invention. That is, the artificial nail composition of the present invention is characterized by containing a polymerizable compound as a component (A), urethane polymer particles as a component (B), and a polymerization initiator as a component (C).

As one embodiment of the artificial nail composition of the present invention, the average particle size of the urethane polymer particles as the component (B) is preferably 0.8 to 52 μm.

As another embodiment of the artificial nail composition of the present invention, the content of the urethane polymer particles as the component (B) is preferably 5 to 50 mass % with respect to the total amount of the artificial nail composition.

As another embodiment of the artificial nail composition of the present invention, it is preferable that the urethane polymer particles as the component (B) have a crosslinked structure.

As another embodiment of the artificial nail composition of the present invention, the polymerizable compound as the component (A) is preferably at least one selected from the group consisting of a urethane (meth)acrylate oligomer and a polymerizable monomer having an ethylenically unsaturated group.

As another embodiment of the artificial nail composition of the present invention, the polymerization initiator as the component (C) is preferably a photopolymerization initiator.

As another embodiment of the artificial nail composition of the present invention, the artificial nail composition preferably contains 49 to 94 mass % of a polymerizable compound as a component (A), 5 to 50 mass % of urethane polymer particles as a component (B), and 0.1 to 10 mass % of a polymerization initiator as a component (C).

As another embodiment of the artificial nail composition of the present invention, the artificial nail composition is preferably a nail polish, a gel nail, or an acrylic nail.

The present invention also relates to an artificial nail obtained from the artificial nail composition.

The present invention also relates to a method for producing an artificial nail, the method including the steps of applying an artificial nail composition containing a polymerizable compound as a component (A), urethane polymer particles as a component (B), and a polymerization initiator as a component (C) onto a nail or a substrate and curing the composition.

The present invention also relates to a method for forming an artificial nail, in which an artificial nail composition containing a polymerizable compound as a component (A), urethane polymer particles as a component (B), and a polymerization initiator as a component (C) is applied onto a nail or a substrate and cured.

The present invention also relates to the use of a composition containing a polymerizable compound as a component (A), urethane polymer particles as a component (B), and a polymerization initiator as a component (C) for the production of an artificial nail composition.

The present invention also relates to the use of a composition containing a polymerizable compound as a component (A), urethane polymer particles as a component (B), and a polymerization initiator as a component (C) for the production of an artificial nail.

According to the present invention, it is possible to provide an artificial nail composition having a matte texture with reduced gloss and having high flexibility.

### DETAILED DESCRIPTION OF THE PREFERRED EMBODIMENTS

The artificial nail composition of the present invention contains a polymerizable compound as a component (A), urethane polymer particles as a component (B), and a polymerization initiator as a component (C).

### [Polymerizable Compound]

The polymerizable compound as the component (A) is a substance that reacts with light or heat to form a polymer. It imparts properties such as curability, surface hardness, strength, flexibility, durability, and removability to the artificial nail composition of the present invention. The polymerizable compound as the component (A) is not particularly limited as long as it is a compound (for example, a polymerizable monomer, an oligomer, or a polymer) having at least one ethylenically unsaturated group as a polymerizable functional group, and a known polymerizable compound can be used. Specific examples of the ethylenically unsaturated group include a (meth)acryloyl group, a (meth)acryloyloxy group, a (meth)acrylamide group, a vinyl group, a vinyl ether group, a methyl vinyl ether group, an allyl group, an allyl ether group, and a maleimide group, but are not limited thereto. One kind of the ethylenically unsaturated group may be contained, or two or more kinds thereof may be contained. Among them, from the viewpoint of curability, surface hardness, and durability, at least one selected from the group consisting of a (meth)acryloyl group and a (meth)acryloyloxy group is preferable.

In the present specification, the term “(meth)acryloyl” encompasses both acryloyl and methacryloyl, the term “(meth)acrylate” encompasses both acrylate and methacrylate, the term “(meth)acryloyloxy” encompasses both acryloyloxy and methacryloyloxy, and the term “(meth)acrylamide” encompasses both acrylamide and methacrylamide.

Specific examples of the polymerizable compound having one ethylenically unsaturated group in one molecule as the component (A) include polymerizable monomers such as methacrylic acid, acrylic acid, urethane (meth)acrylate, methoxyethylene glycol (meth)acrylate, methoxypolyethylene glycol (meth)acrylate, phenoxyethylene glycol (meth)acrylate, phenoxypolyethylene glycol (meth)acrylate, 2-hydroxy-3-phenoxypropyl (meth)acrylate, 2-(meth)acryloyloxyethyl succinate, 2-(meth)acryloyloxyethyl phthalate, 2-(meth)acryloyloxypropyl hexaphthalate, stearyl (meth)acrylate, methyl (meth)acrylate, ethyl (meth)acrylate, hydroxyethyl (meth)acrylate, hydroxypropyl (meth)acrylate, 3-chloro-2-hydroxypropyl (meth)acrylate, isobornyl (meth)acrylate, tetrahydrofurfuryl (meth)acrylate, 2-(meth)acryloyloxyethyl dihydrogen phosphate, 2-(meth)acryloyloxyethyl phenyl hydrogen phosphate, 10-(meth)acryloyloxydecyl dihydrogen phosphate, 6-(meth)acryloyloxyhexyl dihydrogen phosphate, 2-(meth)acryloyloxyethyl 2-bromoethyl hydrogen phosphate, methylol (meth)acrylamide, dimethyl (meth)acrylamide, (meth)acryloyl morpholine, and vinyl chloride; oligomers of the polymerizable monomers; and polymers of the polymerizable monomers, but are not limited thereto. The oligomer and the polymer may contain one or two or more of the polymerizable monomers exemplified above.

The term “oligomer” in the present specification means an oligomer obtained by polymerizing 2 to several tens of polymerizable monomers. The term “polymer” means a polymer obtained by polymerizing several tens or more polymerizable monomers.

Specific examples of the polymerizable compound having two ethylenically unsaturated groups in one molecule as the component (A) include polymerizable monomers such as urethane di(meth)acrylate, 1,6-hexanediol di(meth)acrylate, 1,9-nonanediol di(meth)acrylate, 1,10-decanediol di(meth)acrylate, neopentyl glycol di(meth)acrylate, 2-methyl-1,8-octanediol di(meth)acrylate, glycerin di(meth)acrylate, ethylene glycol di(meth)acrylate, triethylene glycol di(meth)acrylate, polyethylene glycol di(meth)acrylate, propylene glycol di(meth)acrylate, polypropylene glycol di(meth)acrylate, ethoxylated polypropylene glycol di(meth)acrylate, ethoxylated propylene glycol di(meth)acrylate, ethoxylated bisphenol A di(meth)acrylate, propoxylated bisphenol A di(meth)acrylate, propoxylated ethoxylated bisphenol A di(meth)acrylate, tricyclodecane dimethanol di(meth)acrylate, and bis(2-(meth)acryloyloxyethyl) hydrogen phosphate; oligomers of the polymerizable monomers; and polymers of the polymerizable monomers, but are not limited thereto. The oligomer and the polymer may contain one or two or more of the polymerizable monomers exemplified above.

Specific examples of the polymerizable compound having three or more ethylenically unsaturated groups in one molecule as the component (A) include polymerizable monomers such as urethane tri(meth)acrylate, urethane tetra(meth)acrylate, urethane penta(meth)acrylate, urethane hexa(meth)acrylate, trimethylolpropane tri(meth)acrylate, ethoxylated trimethylolpropane tri(meth)acrylate, ethoxylated glycerin tri(meth)acrylate, ditrimethylolpropane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, ethoxylated isocyanurate tri(meth)acrylate, pentaerythritol tetra(meth)acrylate, tetramethylolmethane tetra(meth)acrylate, dipentaerythritol tetra(meth)acrylate, ditrimethylolpropane tetra(meth)acrylate, dipentaerythritol penta(meth)acrylate, caprolactone-modified pentaerythritol penta(meth)acrylate, dipentaerythritol hexa(meth)acrylate, and caprolactone-modified pentaerythritol hexa(meth)acrylate; oligomers of the polymerizable monomers; and polymers of the polymerizable monomers, but are not limited thereto. The oligomer and the polymer may contain one or two or more of the polymerizable monomers exemplified above.

The molecular weight of the polymerizable monomer as the polymerizable compound which is the component (A) is not particularly limited, but is preferably less than 1,000. The weight average molecular weight (Mw) of the oligomer as the polymerizable compound which is the component (A) is not particularly limited, but is, for example, 30,000 or less, preferably 300 to 30,000, more preferably 500 to 25,000, still more preferably 700 to 20,000, and particularly preferably 700 to 18,000. The weight average molecular weight (Mw) of the polymer as the polymerizable compound which is the component (A) is not particularly limited, and is, for example, more than 30,000, preferably more than 30,000 and 60,000 or less. Within this range, properties such as curability, surface hardness, strength, flexibility, durability, and removability can be improved. In the present specification, as the weight average molecular weight (Mw), a value measured by gel permeation chromatography (GPC) using polystyrene as a standard substance, is adopted.

In an atmosphere at 23° C., the polymerizable monomer as the polymerizable compound which is the component (A) is preferably a liquid and has fluidity. Specifically, the polymerizable monomer preferably has a viscosity of 20,000 mPa·s or less, more preferably 5,000 mPa·s or less, still more preferably 1,000 mPa·s or less, and particularly preferably 100 mPa·s or less at 23° C. and a shear rate of 10 s⁻¹ as measured using a rheometer, which is a dynamic viscoelasticity measuring device. The oligomer as the polymerizable compound which is the component (A) may or may not have fluidity in an atmosphere at 23° C. Specifically, the oligomer preferably has a viscosity of 9,000 mPa·s or more, more preferably 9,000 to 3,000,000 mPa·s, still more preferably 9,000 to 2,500,000 mPa·s, and particularly preferably 9,000 to 2,200,000 mPa·s at 23° C. and a shear rate of 10 s⁻¹ as measured using a rheometer, which is a dynamic viscoelasticity measuring device. The polymer as the polymerizable compound which is the component (A) may or may not have fluidity in an atmosphere at 23° C.

Two or more polymerizable compounds as the component (A) can be selected to form an artificial nail composition. Among these examples, it is preferable to use at least one selected from the group consisting of methacrylic acid, acrylic acid, urethane (meth)acrylate, urethane di(meth)acrylate, urethane tri(meth)acrylate, urethane tetra(meth)acrylate, urethane penta(meth)acrylate, urethane hexa(meth)acrylate, isobornyl (meth)acrylate, tetrahydrofurfuryl (meth)acrylate, hydroxypropyl (meth)acrylate, triethylene glycol di(meth)acrylate, (meth) acryloylmorpholine, bis(2-(meth)acryloyloxyethyl) hydrogen phosphate, trimethylolpropane tri(meth)acrylate, oligomers thereof, and polymers thereof. The oligomer and the polymer may contain one or two or more of the polymerizable monomers exemplified above. The polymerizable compound as the component (A) more preferably contains the oligomer and the polymerizable monomer, still more preferably contains at least one oligomer selected from the group consisting of a urethane (meth)acrylate oligomer, a urethane di(meth)acrylate oligomer, and a urethane hexa(meth)acrylate oligomer, and at least one polymerizable monomer selected from the group consisting of isobornyl (meth)acrylate, tetrahydrofurfuryl (meth)acrylate, hydroxypropyl (meth)acrylate, triethylene glycol di(meth)acrylate, (meth) acryloylmorpholine, and trimethylolpropane tri(meth)acrylate, and particularly preferably contains a urethane di(meth)acrylate oligomer and at least one polymerizable monomer selected from the group consisting of isobornyl (meth)acrylate and tetrahydrofurfuryl (meth)acrylate. When the polymerizable compound as the component (A) contains the oligomer and the polymerizable monomer, properties such as flexibility, durability, removability, curability, surface hardness, and strength can be improved.

The content of the polymerizable compound as the component (A) is not particularly limited, but the content of the polymerizable compound as the component (A) with respect to the total amount of the composition is preferably within a range of 49 to 94 mass %, more preferably within a range of 55 to 87 mass %, and still more preferably within a range of 60 to 80 mass %. When the content of the polymerizable compound as the component (A) is in a range of 49 to 94 mass %, properties such as matte texture, flexibility, durability, removability, curability, surface hardness, and strength can be improved.

When the polymerizable compound as the component (A) contains an oligomer and a polymerizable monomer, the content of the polymerizable monomer is usually about 10 to 1000 parts by mass per 100 parts by mass of the oligomer. From the viewpoint of further improving properties such as flexibility, durability, removability, curability, surface hardness, and strength, the content is preferably 50 to 750 parts by mass, more preferably 100 to 750 parts by mass, still more preferably 200 to 650 parts by mass, and further preferably 200 to 550 parts by mass.

### [Urethane Polymer Particles]

The urethane polymer particles as the component (B) form irregularities on the surface of the cured film of the artificial nail composition of the present invention, and cause irregular reflection of light to impart a matte texture. The urethane polymer particles further impart properties such as high flexibility, luxuriousness, strength, and durability to the artificial nail composition of the present invention. The urethane polymer particles as the component (B) are polymer particles having at least one urethane bond in a molecular structure thereof. The production method is not particularly limited, but they can be obtained, for example, by reacting a polyisocyanate having two or more isocyanate groups with a polyol having two or more hydroxy groups.

Specific examples of the polyisocyanate include diisocyanates such as hexamethylene diisocyanate, 2,4-diisocyanate-1-methylcyclohexane, diisocyanate cyclobutane, tetramethylene diisocyanate, o-, m- or p-xylylene diisocyanate, hydrogenated xylylene diisocyanate, dicyclohexylmethane diisocyanate, dimethyldicyclohexylmethane diisocyanate, lysine diisocyanate, cyclohexane diisocyanate, dodecane diisocyanate, tetramethylxylene diisocyanate, isophorone diisocyanate, tolylene-2,4-diisocyanate, tolylene-2,6-diisocyanate, diphenylmethane-4,4′-diisocyanate, 3-methyldiphenylmethane-4,4′-diisocyanate, m- or p-phenylene diisocyanate, chlorophenylene-2,4-diisocyanate, naphthaline-1,5-diisocyanate, diphenyl-4,4′-diisocyanate, 3,3′-dimethyldiphenyl-1,3,5-triisopropylbenzene-2,4-diisocyanate, carbodiimide-modified diphenylmethane diisocyanate, and diphenyl ether diisocyanate; and tri- or higher functional polyisocyanates such as 2,4,6-triisocyanate toluene, 1,3,5-triisocyanate benzene, triphenylmethane triisocyanate, and polyphenyl polymethylene isocyanate, but are not limited thereto. These polyisocyanates may be used singly or in combination of two or more kinds thereof.

Specific examples of the polyol include diols such as ethylene glycol, propylene glycol, 1,3-propanediol, 1,4-butanediol, 1,3-butanediol, 1,5-pentanediol, neopentyl glycol, 1,6-hexanediol, 3-methyl-1,5-pentanediol, 1,4-bis(hydroxymethyl) cyclohexane, bisphenol A, hydrogenated bisphenol A, hydroxypivalyl hydroxypivalate, 2,2,4-trimethyl-1,3-pentanediol, polyether glycol, polyoxyethylene glycol, polyoxypropylene glycol, polyoxyethylene polyoxytetramethylene glycol, polyoxypropylene polyoxytetramethylene glycol, polyoxyethylene polyoxypropylene polyoxytetramethylene glycol, modified polyether diols obtained by ring-opening polymerization of these dihydric alcohols with cyclic ether bond-containing compounds, and polyester diols obtained by condensation polymerization of one or more of these dihydric alcohols with a dicarboxylic acid; and trihydric or higher polyols such as trimethylolethane, trimethylolpropane, glycerin, hexanetriol, ditrimethylolpropane, pentaerythritol, dipentaerythritol, tripentaerythritol, diglycerol, triglycerol, tris(2-hydroxyethyl) isocyanurate, cyclohexanetriol, trihydroxynaphthalene, tetrahydroxynaphthalene, benzenetriol, biphenyltetraol, modified polyether polyols obtained by ring-opening polymerization of these trihydric or higher alcohols with cyclic ether bond-containing compounds, and polyester polyols obtained by condensation polymerization of one or more of these trihydric or higher alcohols with a polyvalent carboxylic acid, but are not limited thereto. These polyols may be used singly or in combination of two or more kinds thereof.

The polymerization method for obtaining the urethane polymer particles as the component (B) is not particularly limited. Specific examples of the polymerization method include emulsion polymerization, seed polymerization, and suspension polymerization. In addition, the shape of the urethane polymer particles as the component (B) is not particularly limited, and particles having any particle shape such as a spherical shape, a needle shape, a plate shape, a crushed shape, or a scaly shape can be used without any limitation. Among the examples described above, a spherical shape is preferable.

The macromolecule structure of the urethane polymer particles as the component (B) is not particularly limited as long as the urethane polymer particles are not dissolved in other components and can impart a matte texture to the artificial nail composition of the present invention, and a linear macromolecule, a macromolecule having a branched structure such as a star-shaped macromolecule, a comb-shaped macromolecule, a brush-shaped macromolecule, or a crosslinked macromolecule, or a combination thereof can be used. Among them, urethane polymer particles having a crosslinked structure are preferable from the viewpoint of being less likely to dissolve in other components other than the urethane polymer particles as the component (B). The crosslinked structure of the urethane polymer particles can be formed by, for example, a method using a tri- or higher functional polyisocyanate, a method using a tri- or higher valent polyol, or a method using these in combination.

The average particle size of the urethane polymer particles as the component (B) is not particularly limited, but is preferably within a range of 0.8 to 52 μm, more preferably within a range of 5 to 52 μm, and still more preferably within a range of 5 to 25 μm.

The term “average particle size” in the present specification means a value of the particle size at a cumulative volume of 50% obtained by measuring a particle size distribution with a laser diffraction particle size distribution analyzer.

The particle size at a cumulative volume of 10% of the urethane polymer particles as the component (B) is not particularly limited, but is preferably within a range of 0.1 to 20 μm, more preferably within a range of 1 to 18 μm, and still more preferably within a range of 1 to 16 μm.

The particle size at a cumulative volume of 90% of the urethane polymer particles as the component (B) is not particularly limited, but is preferably within a range of 3 to 90 μm, more preferably within a range of 4 to 70 μm, and still more preferably within a range of 5 to 50 μm.

The glass transition temperature Tg of the urethane polymer particles as the component (B) is not particularly limited, but is preferably −60° C. to 0° C., and more preferably −20° C. to 0° C. When the temperature is in a range of −60° C. to 0° C., high flexibility can be imparted.

The glass transition temperature Tg in the present specification means a midpoint glass transition temperature measured by a differential scanning calorimetry (DSC) method. In addition, the term “midpoint glass transition temperature” in the present specification means a temperature at a point where a straight line equidistant in the vertical axis direction from the extended straight line of each baseline intersects the curve of the stepwise change portion of the glass transition.

The oil absorption amount of the urethane polymer particles as the component (B) is not particularly limited, but is preferably 1 g/g to 5 g/g, and more preferably 1.5 g/g to 4.5 g/g. When the content is in a range of 1 g/g to 5 g/g, the storage stability of the artificial nail composition of the present invention can be improved.

The oil absorption amount in the present specification means a value measured by the following method. That is, after 40 g of the urethane polymer particles are put into a container, 5.0 g of toluene is poured. While stirring the sample in the container using a stirring rod, toluene is additionally added until the sample becomes flowable, and then the sample is left to stand for 1 hour. When the sample maintains fluidity after standing, the total mass of toluene added until this point is taken as the oil absorption amount. When the fluidity of the sample disappears after standing, toluene is further added until the sample becomes flowable, and the total mass of toluene added until this point is taken as the oil absorption amount. Here, the fluidity is a state in which when the stirring rod used for stirring the sample is pulled up, the sample attached to the stirring rod falls within 10 seconds after the pulling up.

The content of the urethane polymer particles as the component (B) is not particularly limited, but is preferably within a range of 5 to 50 mass %, more preferably 11 to 44 mass %, and still more preferably 18 to 39 mass % with respect to the total amount of the composition. When the content is in a range of 5 to 50 mass %, matting properties, flexibility, luxuriousness, and good operability can be imparted.

### [Polymerization Initiator]

The polymerization initiator as the component (C) is a compound for initiating a polymerization reaction of the polymerizable compound as the component (A). The kind of the polymerization initiator (C) is not particularly limited, and a known polymerization initiator can be used. Examples of the polymerization initiator as the component (C) include a photopolymerization initiator that serves as a starting point of a polymerization reaction by irradiation with energy rays such as visible rays, ultraviolet rays, X-rays, or electron beams, a thermal polymerization initiator that serves as a starting point of a polymerization reaction by heating to a certain temperature or higher, and a chemical polymerization initiator that serves as starting points of a polymerization reaction by mixing specific substances and is a type in which two or more agents are mixed, and a photopolymerization initiator is preferable. Examples of the photopolymerization initiator include a radical polymerization initiator that generates a radical by irradiation with energy rays, a cationic polymerization initiator that generates a cation, and an anionic polymerization initiator that generates an anion, and a radical polymerization initiator is preferable. Specific examples of the radical photopolymerization initiator include benzoin ethers, benzyl ketals, α-dialkoxyacetophenones, α-hydroxyalkylphenones, α-aminoalkylphenones, acylphosphine oxides, benzophenones, thioxanthones, and titanocenes, but are not limited thereto. Two or more of these polymerization initiators can be selected to form an artificial nail composition. Among them, it is preferable to use at least one selected from the group consisting of α-hydroxyalkylphenones and acylphosphine oxides.

Specific examples of the α-hydroxyalkylphenones include 1-hydroxycyclohexyl phenyl ketone, 2-hydroxy-2-methyl-1-phenyl-propane-1-one, and α-aminoalkylphenone. Specific examples of the acylphosphine oxides include diphenyl(2,4,6-trimethylbenzoyl) phosphine oxide and bis(2,4,6-trimethylbenzoyl)phenylphosphine oxide. Among them, it is preferable to use at least one selected from diphenyl(2,4,6-trimethylbenzoyl) phosphine oxide and bis(2,4,6-trimethylbenzoyl)phenylphosphine oxide.

The content of the polymerization initiator as the component (C) is not particularly limited, but is preferably in a range of 0.1 to 10 mass %, more preferably in a range of 0.1 to 6 mass %, and still more preferably in a range of 1 to 6 mass % with respect to the total amount of the composition. When the content is in a range of 0.1 to 10 mass %, properties such as curability, surface hardness, and durability can be improved.

### [Other Components]

The artificial nail composition of the present invention may contain components other than the components (A) to (C) as long as the effects of the present invention are not impaired. Examples of the other components include solvents, auxiliary agents, additives, colorants, leveling agents, plasticizers, antioxidants, polymerization accelerators, polymerization inhibitors, coalescing agents, preservatives, waxes, thickeners, fragrances, UV shielding agents, diffusers, defoamers, dispersants, fillers, surfactants, pigments, dyes, excipients, ion releasing agents, and silane coupling agents, which are widely used in artificial nail compositions.

As the polymerization accelerator, a polyfunctional thiol compound having two or more thiol groups in one molecule can be used. By using the polyfunctional thiol compound, the curing reaction proceeds without being inhibited by oxygen. Examples of specific compounds as the polyfunctional thiol compound include 1,2-ethanedithiol, 1,2-propanedithiol, 1,3-propanedithiol, 1,3-butanedithiol, 2,3-butanedithiol, 1,5-pentanedithiol, 1,6-hexanedithiol, 1,8-octanedithiol, 1,9-nonanedithiol, 1,10-decanedithiol, 1,2-benzenedithiol, 1,3-benzenedithiol, 1,4-benzenedithiol, 3,6-dichloro-1,2-benzenedithiol, toluene-3,4-dithiol, 1,5-naphthalenedithiol, ethylene glycol bis(thioglycolate), ethylene glycol bis(3-mercaptopropionate), 1,4-butanediol bisthioglycolate, tetraethylene glycol bis(3-mercaptopropionate), trimethylolpropane tris(thioglycolate), trimethylolpropane tris(3-mercaptopropionate), trimethylolpropane tris(3-mercaptobutyrate), tris [(3-mercaptopropionyloxy)-ethyl] isocyanurate, pentaerythritol tetrakis(thioglycolate), pentaerythritol tetrakis(3-mercaptopropionate), dipentaerythritol hexakis (3-mercaptopropionate), 1,4-bis(3-mercaptobutyryloxy) butane, pentaerythritol tetrakis(3-mercaptobutyrate), pentaerythritol tetrakis(3-mercaptobutyrate), 1,3,5-tris(3-mercaptobutyloxyethyl)-1,3,5-triazine-2,4,6 (1H,3H,5H)-trione, dimercaptodiethyl sulfide, 1,8-dimercapto-3,6-dithiaoctane, 1,2-bis [(2-mercaptoethyl)thio]-3-mercaptopropane, tetrakis(7-mercapto-2,5-dithiaheptyl) methane, trithiocyanuric acid, 1,2-benzenedimethanethiol, 4,4′-thiobisbenzenethiol, 2-di-n-butylamino-4,6-dimercapto-s-triazine, 2,5-dimercapto-1,3,4-thiadiazole, 1,8-dimercapto-3,6-dioxaoctane, 1,5-dimercapto-3-thiapentane, tris(2-hydroxyethyl) isocyanurate trimercaptopropionate, 1,4-dimethylmercaptobenzene, 2,4,6-trimercapto-s-triazine, 2-(N,N-dibutylamino)-4,6-dimercapto-s-triazine, bis(4-(2-mercaptopropoxy)phenyl) methane, 1,1-bis(4-(2-mercaptopropoxy)phenyl) ethane, 2,2-bis(4-(2-mercaptopropoxy)phenyl) propane, 2,2-bis(4-(2-mercaptopropoxy)phenyl) butane, 1,1-bis(4-(2-mercaptopropoxy)phenyl) isobutane, 2,2-bis(4-(2-mercaptopropoxy)-3-methylphenyl) propane, 2,2-bis(4-(2-mercaptopropoxy)-5-methylphenyl) propane, bis(2-(2-mercaptopropoxy)-5-methylphenyl) methane, 2,2-bis(4-(2-mercaptopropoxy)-3-t-butylphenyl) propane, tris(4-(2-mercaptopropoxy)phenyl) methane, 1,1,1-tris(4-(2-mercaptopropoxy)phenyl) ethane, bis(4-(2-mercaptobutoxy)phenyl) methane, 2,2-bis(4-(2-mercaptobutoxy)phenyl) propane, tris(4-(2-mercaptobutoxy)phenyl) methane, 1,3,5-triazine-2,4,6-trithiol, and alkyl vinyl ether adducts thereof. However, the present invention is not limited thereto. These polymerization accelerators may be used singly or in combination of two or more kinds thereof.

The artificial nail composition of the present invention can be applied to a natural nail; a nail on which a coating film is formed by an artificial nail composition; and a substrate such as an artificial resin chip, a resin film, or a resin sheet, but is not limited thereto. Examples of the method of application include, but are not limited to, a method of application with a brush, a sponge, a spray, an inkjet, an air knife, a roll, or the like.

The viscosity of the artificial nail composition of the present invention is not particularly limited, but from the viewpoint of operability, the artificial nail composition preferably has fluidity in an atmosphere at 23° C. Specifically, the viscosity at 23° C. and a shear rate of 10 s⁻¹ measured using a rheometer, which is a dynamic viscoelasticity measuring device, is preferably 200,000 mPa·s or less, more preferably 150,000 mPa·s or less, still more preferably 120,000 mPa·s or less, further preferably 100,000 mPa·s or less, and particularly preferably 50,000 mPa·s or less. On the other hand, the lower limit of the viscosity is not particularly limited, but is preferably 1 mPa·s or more, more preferably 1,000 mPa·s or more, and still more preferably 5,000 mPa·s or more. The viscosity of the artificial nail composition of the present invention can be changed by appropriately adjusting the kinds and contents of the components (A) to (C).

The form of the artificial nail composition of the present invention is not particularly limited, and may be a nail polish, a gel nail, an acrylic nail, or the like. Nail polishes are coating materials for nails called nail lacquer, nail enamel, manicure, or the like, and are compositions for producing coating films excellent in aesthetic appearance by drying a contained solvent. Gel nails are agents containing a resin component that is cured by ultraviolet rays or visible rays, and are compositions that are applied onto natural nails or artificial nails and then cured by irradiation with ultraviolet rays or visible rays to form coating films excellent in aesthetic appearance. Acrylic nails are powder-liquid type agents containing polymer beads and a polymerizable monomer, and are compositions which are cured by polymerization of the polymerizable monomer initiated by a peroxide contained in the polymer beads after powder-liquid mixing. The acrylic nails are characterized in that they can not only be applied but also be produced in built-up forms, and are mainly used for extending nails in many cases. Among these, the artificial nail composition of the present invention is preferably provided in the form of a gel nail. By adopting the form of a gel nail, it is possible to provide an artificial nail composition having a matte texture with reduced gloss and having high flexibility.

In the gel nail, a base layer, a color layer, and a topcoat layer are generally laminated in this order on a natural nail or a substrate to form a coating film. The application of the artificial nail composition of the present invention is not particularly limited, and the artificial nail composition may be any layer of a base layer, a color layer, and a topcoat layer. The artificial nail composition of the present invention is preferably used as a topcoat layer from the viewpoint that a matte texture with reduced gloss can be most remarkably exhibited.

Examples of the gel nail include a gel nail for a hand applied to a nail of a hand, a gel nail for a foot applied to a nail of a foot, and a gel nail for an animal applied to a nail of an animal. The use of the artificial nail composition of the present invention is not particularly limited, and the artificial nail composition can be used for any of hands, feet, animals, and the like.

The artificial nail of the present invention is obtained from the artificial nail composition of the present invention. The artificial nail of the present invention can be formed, for example, by applying the artificial nail composition of the present invention to a natural nail, a nail on which a coating film is formed by an artificial nail composition, or a substrate such as an artificial resin chip, a resin film, or a resin sheet, and curing the composition. The method of application is not particularly limited, and examples thereof include a method of application with a brush, a sponge, a spray, an inkjet, an air knife, a roll, or the like. The curing method is not particularly limited, and examples thereof include a method of curing by irradiation with ultraviolet rays or visible rays and a method of curing by heating, and from the viewpoint of rapid curing, a method of curing by irradiation with ultraviolet rays or visible rays is preferred.

Hereinafter, the examples and the comparative examples of the present invention will be specifically described, but the present invention is not limited to these examples.

### [Components Used for Preparation of Artificial Nail Composition]

The components used for preparing the artificial nail compositions of the examples and the comparative examples are shown in Table 1. As the viscosity in Table 1, a value measured under the conditions of 23° C. and a shear rate of 10 s⁻¹ using a rheometer (manufactured by Anton Paar GmbH, trade name: MCR 301), which is a dynamic viscoelasticity measuring device, was adopted. As the weight average molecular weight (Mw) in Table 1, a value measured by gel permeation chromatography (GPC) using a GPC analyzer (manufactured by Shimadzu Corporation, trade name: Nexera GPC system), a column (manufactured by Waters Corporation, trade name: Styragel HR), tetrahydrofuran as an eluent, and polystyrene as a standard substance, was adopted. As the average particle size in Table 1, a value of the particle size at a cumulative volume of 50% determined by measuring a particle size distribution with a laser diffraction particle size distribution analyzer (MicrotracBEL Corp., trade name: Microtrac MT3300EX II), was adopted. The glass transition temperature Tg in Table 1 is a midpoint glass transition temperature measured by a differential scanning calorimetry (DSC) method, and a temperature at a point where a straight line equidistant in the vertical axis direction from the extended straight line of each baseline intersects the curve of the stepwise change portion of the glass transition was adopted as the glass transition temperature Tg. The oil absorption amount in Table 1 is a value measured by the following method. As a sample, 40 g of urethane polymer particles were put in a container, and then 5.0 g of toluene was poured. While stirring the sample in the container using a stirring rod, toluene was additionally added until the sample became flowable, and then the sample was left to stand for 1 hour. When the sample maintained fluidity after standing, the total mass of toluene added until this point was taken as the oil absorption amount. When the fluidity of the sample disappeared after standing, toluene was further added until the sample became flowable, and the total mass of toluene added until this point was taken as the oil absorption amount. The fluidity is a state in which when the stirring rod used for stirring the sample is pulled up, the sample attached to the stirring rod falls within 10 seconds after the pulling up.

TABLE 1

| | | | | Weight | | Glass | |
|---|---|---|---|---|---|---|---|
| | | | | average | Average | transition | Oil |
| | | | | molecular | particle | temperature | absorption |
| | | | Viscosity | weight | size | Tg | amount |
| Component | | | (mPa · s) | (Mw) | (μm) | (° C.) | (g/g) |
| (A) | Polymerizable | Urethane diacrylate | 2,000,000 | 16,000 | — | — | — |
| Polymerizable | compound 1 | oligomer | | | | | |
| compound | Polymerizable | Urethane diacrylate | 703,000 | 6,000 | — | — | — |
| | compound 2 | oligomer | | | | | |
| | Polymerizable | Urethane diacrylate | 9,300 | 740 | — | — | — |
| | compound 3 | oligomer | | | | | |
| | Polymerizable | Tetrahydrofurfuryl | 2.8 | — | — | — | — |
| | compound 4 | acrylate | | | | | |
| | Polymerizable | Izobornyl | 13 | — | — | — | — |
| | compound 5 | methacrylate | | | | | |
| (B) Urethane | Urethane polymer | Crosslinked | — | — | 1 | −13 | 4.3 |
| polymer | particles 1 | particles | | | | | |
| particles | Urethane polymer | Crosslinked | — | — | 6 | −13 | 2.1 |
| | particles 2 | particles | | | | | |
| | Urethane polymer | Crosslinked | — | — | 22 | −13 | 1.6 |
| | particles 3 | particles | | | | | |
| | Urethane polymer | Crosslinked | — | — | 50 | −13 | 1.6 |
| | particles 4 | particles | | | | | |
| | Urethane polymer | Crosslinked | — | — | 75 | −13 | 1.5 |
| | particles 5 | particles | | | | | |
| (B)′ Acrylic | Acrylic polymer | Crosslinked | — | — | 2.2 | — | — |
| polymer | particles 1 | particles | | | | | |
| particles | Acrylic polymer | Crosslinked | — | — | 8 | — | — |
| | particles 2 | particles | | | | | |
| | Acrylic polymer | Crosslinked | — | — | 50 | — | — |
| | particles 3 | particles | | | | | |
| | Acrylic polymer | Crosslinked | — | — | 110 | — | — |
| | particles 4 | particles | | | | | |
| (C) Polymerization | Polymerization | Diphenyl(2,4,6- | — | — | — | — | — |
| initiator | initiator 1 | trimethylbenzoyl)phosphine | | | | | |
| | | oxide | | | | | |
| | Polymerization | Bis(2,4,6- | — | — | — | — | — |
| | initiator 2 | trimethylbenzoyl)phenylphosphine | | | | | |
| | | oxide | | | | | |

### [Preparation of Photocurable Artificial Nail Composition]

The amount of each component was calculated according to each blending ratio described in Tables 3 to 6, and the components were mixed until a homogeneous liquid was obtained using a planetary centrifugal mixer (manufactured by THINKY CORPORATION, trade name: ARV-310P) under atmospheric pressure to prepare each artificial nail composition of the examples and the comparative examples.

Evaluation methods of the artificial nail compositions of the examples and the comparative examples are as follows. The evaluation was performed under indoor LED illumination at a room temperature of 23±2° C. and a humidity of 50±10% unless otherwise specified.

### Test Example 1: Evaluation of Matte Texture

Each artificial nail composition of the examples and the comparative examples was applied onto a glass flat plate using a spacer, and cured by light irradiation with a commercially available gel nail light (manufactured by Nail Labo Corporation, trade name: PRESTO LED light) for 20 seconds, and then a surface uncured layer portion of the artificial nail composition was removed using a wipe soaked in ethanol to prepare a thin film having a thickness of 0.2 mm, a long side of 50.0 mm, and a short side of 15.0 mm. Next, 60° gloss was measured using a gloss meter (manufactured by HORIBA, Ltd., model: HORIBA Gloss Checker IG-331). In addition, three thin films were prepared for one artificial nail composition, and the matte texture was evaluated. The value of the matte texture evaluation is an average value of measured values of 60° gloss measured using three thin films, and when the value is 15.0 or less, it is determined that the film has a matte texture, and when the value is 10.0 or less, it is preferable, and when the value is 5.0 or less, it is more preferable.

### Test Example 2: Viscosity Measurement

The artificial nail compositions of the examples and the comparative examples were subjected to viscosity measurement under the following measurement conditions using a rheometer (manufactured by Anton Paar GmbH, model: Physica MCR 301), which is a dynamic viscoelasticity measuring device, and the measured value at a shear rate of 10 s⁻¹ was taken as the viscosity value of each artificial nail composition of the examples and the comparative examples. From the viewpoint of operability, the viscosity value of the artificial nail composition is preferably 200,000 mPa·s or less, more preferably 150,000 mPa·s or less, still more preferably 120,000 mPa·s or less, further preferably 100,000 mPa·s or less, and particularly preferably 50,000 mPa·s or less. On the other hand, the lower limit of the viscosity is not particularly limited, but is preferably 1 mPa·s or more, more preferably 1,000 mPa·s or more, and still more preferably 5,000 mPa·s or more.

### (Measurement Conditions)

Measurement jig: Parallel plate PP20
Measurement temperature: 23° C.
Shear rate: 0.1 to 100 s⁻¹

### Test Example 3: Measurement of Elongation at Break

The artificial nail compositions of the examples and the comparative examples were cured by light irradiation with a commercially available gel nail light (manufactured by Nail Labo Corporation, trade name: PRESTO LED light) for 20 seconds to prepare dumbbell-shaped test pieces having a thickness of 1.9 mm, a total length of 28.6 mm, a distance between tabs of 25.0 mm, a length of the parallel part of 15.0 mm, a radius of the shoulder part of 7.0 mm, and a width of the parallel part of 2.0 mm, and these test pieces were used as test pieces for measuring elongation at break. After standing all day and night, the elongation at break was measured using an Instron universal tester (manufactured by Instron Corporation, model: Instron 5943 type) under the condition of a crosshead speed of 10 mm/min. When the value of the elongation at break is 25.0 [%] or more, it is determined that the film has high flexibility, and when the value is 30.0 [%] or more, it is preferable, and when the value is 35.0 [%] or more, it is more preferable.

### Test Example 4: Tactile Sensation Test

Each artificial nail composition of the examples and the comparative examples was applied onto a glass flat plate using a spacer, and cured by light irradiation with a commercially available gel nail light (manufactured by Nail Labo Corporation, trade name: PRESTO LED light) for 20 seconds, and then a surface uncured layer portion of the artificial nail composition was removed using tissue paper soaked in ethanol to prepare a thin film having a thickness of 0.2 mm, a long side of 50.0 mm, and a short side of 15.0 mm. Next, 10 nail technicians certified by the Japan Nailist Association touched the light-irradiated surface of the thin film with their fingertips cleaned with ethanol to perform sensory evaluation of the tactile sensation of the thin film, brought the evaluation results according to the criteria shown in Table 2, and comprehensively evaluated. From the viewpoint of tactile quality, the comprehensive evaluation result is preferably A.

TABLE 2

| | Evaluation |
|---|---|
| Evaluation criteria | results |
| It had a moist and soft touch and felt luxurious. | A |
| It had a smooth touch, and did not feel luxurious. | B |
| It had a rough touch as if it was caught by the fingertip, | C |
| and was unpleasant to the touch. | |
| It had a slippery touch without a matte texture, and did | D |
| not feel luxurious. | |

### Evaluation Results of Examples and Comparative Examples

Tables 3 to 6 show the evaluation results of the matte texture, viscosity, elongation at break, and tactile sensation of each of the examples and the comparative examples.

TABLE 3

| | | | | | | | | | | Compar- |
|---|---|---|---|---|---|---|---|---|---|---|
| | | Example | Example | Example | Example | Example | Example | Example | Example | ative |
| | | 1 | 2 | 3 | 4 | 5 | 6 | 7 | 8 | Example 1 |
| (A) | Polymerizable | | | | | | | | | |
| Polymerizable | compound 1 | | | | | | | | | |
| compound | Polymerizable | 7.3 | 15.4 | 17.2 | 19.0 | 20.6 | 25.0 | 14.2 | 16.4 | 17.2 |
| | compound 2 | | | | | | | | | |
| | Polymerizable | | | | | | | | | |
| | compound 3 | | | | | | | | | |
| | Polymerizable | 21.9 | 17.6 | 19.8 | 21.9 | 23.7 | 28.7 | 42.8 | 45.8 | 48.3 |
| | compound 4 | | | | | | | | | |
| | Polymerizable | 15.3 | 16.2 | 18.1 | 20.0 | 21.6 | 26.3 | 29.9 | 31.1 | 32.8 |
| | compound 5 | | | | | | | | | |
| (B) Urethane | Urethane | | | | | | | | | |
| polymer | polymer | | | | | | | | | |
| particles | particles 1 | | | | | | | | | |
| | Urethane | 32.9 | 30.8 | 31.0 | 27.6 | 26.8 | 15.0 | 11.7 | 5.1 | |
| | polymer | | | | | | | | | |
| | particles 2 | | | | | | | | | |
| | Urethane | 21.9 | 19.2 | 13.0 | 10.5 | 6.3 | 3.7 | | | |
| | polymer | | | | | | | | | |
| | particles 3 | | | | | | | | | |
| | Urethane | | | | | | | | | |
| | polymer | | | | | | | | | |
| | particles 4 | | | | | | | | | |
| | Urethane | | | | | | | | | |
| | polymer | | | | | | | | | |
| | particles 5 | | | | | | | | | |
| (B)′ Acrylic | Acrylic | | | | | | | | | |
| polymer | polymer | | | | | | | | | |
| particles | particles 1 | | | | | | | | | |
| | Acrylic | | | | | | | | | |
| | polymer | | | | | | | | | |
| | particles 2 | | | | | | | | | |
| | Acrylic | | | | | | | | | |
| | polymer | | | | | | | | | |
| | particles 3 | | | | | | | | | |
| | Acrylic | | | | | | | | | |
| | polymer | | | | | | | | | |
| | particles 4 | | | | | | | | | |
| (C) | Polymerization | 0.7 | 0.8 | 0.9 | 1.0 | 1.0 | 1.3 | 1.4 | 1.6 | 1.7 |
| Polymerization | initiator 1 | | | | | | | | | |
| initiator | Polymerization | | | | | | | | | |
| | initiator 2 | | | | | | | | | |
| | Total (mass %) | 100.0 | 100.0 | 100.0 | 100.0 | 100.0 | 100.0 | 100.0 | 100.0 | 100.0 |

| | | | | | | | | | |
|---|---|---|---|---|---|---|---|---|---|
| Test Example 1: Evaluation | 0 | 0 | 0 | 0 | 0 | 0 | 9 | 14 | 98 |
| of matte texture | | | | | | | | | |
| Test Example 2: Viscosity measurement | 382000 | 196000 | 89000 | 45000 | 28000 | 12000 | 7000 | 5000 | 3000 |
| (mPa · s) | | | | | | | | | |
| Test Example 3: Measurement of | 70.0 | 64.0 | 55.0 | 51.0 | 40.0 | 38.0 | 32.0 | 26.0 | 15.0 |
| elongation at break (%) | | | | | | | | | |
| Test Example 4: Tactile | A | A | A | A | A | A | A | A | D |
| sensation test | | | | | | | | | |

TABLE 4

| | | Example | Example | Example | Example | Example | Example | Example | Example | Example |
|---|---|---|---|---|---|---|---|---|---|---|
| | | 9 | 10 | 11 | 12 | 13 | 14 | 15 | 16 | 17 |
| (A) | Polymerizable | 6.9 | | 5.2 | | 3.5 | | | | |
| Polymerizable | compound 1 | | | | | | | | | |
| compound | Polymerizable | | | 5.2 | 16.7 | 8.1 | 21.1 | 21.1 | 21.1 | 21.1 |
| | compound 2 | | | | | | | | | |
| | Polymerizable | | 26.8 | | 16.7 | 14.0 | | | | |
| | compound 3 | | | | | | | | | |
| | Polymerizable | 55.6 | | 41.2 | 16.7 | 34.9 | 24.2 | 24.2 | 24.2 | 24.2 |
| | compound 4 | | | | | | | | | |
| | Polymerizable | | 26.8 | 21.6 | 35.0 | 9.3 | 22.0 | 22.0 | 22.0 | 22.0 |
| | compound 5 | | | | | | | | | |
| (B) Urethane | Urethane polymer | | | | | | 31.6 | | | |
| polymer | particles 1 | | | | | | | | | |
| particles | Urethane polymer | 36.1 | 32.1 | | 13.2 | 29.0 | | 31.6 | | |
| | particles 2 | | | | | | | | | |
| | Urethane polymer | | 13.4 | 25.8 | | | | | 31.6 | |
| | particles 3 | | | | | | | | | |
| | Urethane polymer | | | | | | | | | 31.6 |
| | particles 4 | | | | | | | | | |
| | Urethane polymer | | | | | | | | | |
| | particles 5 | | | | | | | | | |
| (B)′ Acrylic | Acrylic polymer | | | | | | | | | |
| polymer | particles 1 | | | | | | | | | |
| particles | Acrylic polymer | | | | | | | | | |
| | particles 2 | | | | | | | | | |
| | Acrylic polymer | | | | | | | | | |
| | particles 3 | | | | | | | | | |
| | Acrylic polymer | | | | | | | | | |
| | particles 4 | | | | | | | | | |
| (C) | Polymerization | 1.4 | 0.9 | 1.0 | 1.7 | 1.2 | 1.1 | 1.1 | 1.1 | 1.1 |
| Polymerization | initiator 1 | | | | | | | | | |
| initiator | Polymerization | | | | | | | | | |
| | initiator 2 | | | | | | | | | |
| | Total (100 mass %) | 100.0 | 100.0 | 100.0 | 100.0 | 100.0 | 100.0 | 100.0 | 100.0 | 100.0 |

| | | | | | | | | | |
|---|---|---|---|---|---|---|---|---|---|
| Test Example 1: Evaluation of | 2 | 0 | 0 | 8 | 0 | 0 | 0 | 0 | 12 |
| matte texture | | | | | | | | | |
| Test Example 2: Viscosity measurement | 32000 | 188000 | 20000 | 6000 | 48000 | 102000 | 35000 | 31000 | 28000 |
| (mPa · s) | | | | | | | | | |
| Test Example 3: Measurement of | 36.0 | 55.0 | 51.0 | 29.0 | 37.0 | 71.0 | 47.0 | 38.0 | 28.0 |
| elongation at break (%) | | | | | | | | | |
| Test Example 4: Tactile | A | A | A | A | A | A | A | A | A |
| sensation test | | | | | | | | | |

TABLE 5

| | | | Compar- | Compar- | Compar- | Compar- | | | | |
|---|---|---|---|---|---|---|---|---|---|---|
| | | | ative | ative | ative | ative | Example | Example | Example | Example |
| | | Example 18 | Example 2 | Example 3 | Example 4 | Example 5 | 19 | 20 | 21 | 22 |
| (A) | Polymerizable | | | | | | | | | |
| Polymerizable | compound 1 | | | | | | | | | |
| compound | Polymerizable | 21.1 | 21.1 | 21.1 | 21.1 | 21.1 | 21.1 | 21.1 | 21.1 | 21.1 |
| | compound 2 | | | | | | | | | |
| | Polymerizable | | | | | | | | | |
| | compound 3 | | | | | | | | | |
| | Polymerizable | 24.2 | 24.2 | 24.2 | 24.2 | 24.2 | 24.2 | 24.2 | 24.2 | 24.2 |
| | compound 4 | | | | | | | | | |
| | Polymerizable | 22.0 | 22.0 | 22.0 | 22.0 | 22.0 | 22.0 | 22.0 | 22.1 | 22.1 |
| | compound 5 | | | | | | | | | |
| (B) Urethane | Urethane polymer | | | | | | | | | |
| polymer | particles 1 | | | | | | | | | |
| particles | Urethane polymer | | | | | | 26.3 | | 10.5 | |
| | particles 2 | | | | | | | | | |
| | Urethane polymer | | | | | | | 10.5 | 10.5 | 21.1 |
| | particles 3 | | | | | | | | | |
| | Urethane polymer | | | | | | 5.3 | 21.1 | 10.5 | |
| | particles 4 | | | | | | | | | |
| | Urethane polymer | 31.6 | | | | | | | | |
| | particles 5 | | | | | | | | | |
| (B)′ Acrylic | Acrylic polymer | | 31.6 | | | | | | | |
| polymer | particles 1 | | | | | | | | | |
| particles | Acrylic polymer | | | 31.6 | | | | | | 10.4 |
| | particles 2 | | | | | | | | | |
| | Acrylic polymer | | | | 31.6 | | | | | |
| | particles 3 | | | | | | | | | |
| | Acrylic polymer | | | | | 31.6 | | | | |
| | particles 4 | | | | | | | | | |
| (C) | Polymerization | 1.1 | 1.1 | 1.1 | 1.1 | 1.1 | 1.1 | 1.1 | 1.1 | 1.1 |
| Polymerization | initiator 1 | | | | | | | | | |
| initiator | Polymerization | | | | | | | | | |
| | initiator 2 | | | | | | | | | |
| | Total (100 mass %) | 100.0 | 100.0 | 100.0 | 100.0 | 100.0 | 100.0 | 100.0 | 100.0 | 100.0 |

| | | | | | | | | | |
|---|---|---|---|---|---|---|---|---|---|
| Test Example 1: Evaluation of | 15 | 4 | 6 | 15 | 46 | 0 | 0 | 0 | 0 |
| matte texture | | | | | | | | | |
| Test Example 2: Viscosity measurement | 28000 | 32000 | 33000 | 27000 | 20000 | 33000 | 31000 | 28000 | 25000 |
| (mPa · s) | | | | | | | | | |
| Test Example 3: Measurement of | 26.0 | 9.0 | 7.1 | 10.2 | 9.3 | 40.0 | 36.0 | 36.0 | 30.5 |
| elongation at break (%) | | | | | | | | | |
| Test Example 4: Tactile | C | B | B | B | C | A | A | A | A |
| sensation test | | | | | | | | | |

TABLE 6

| | | Comparative | | | | | |
|---|---|---|---|---|---|---|---|
| | | Example 6 | Example 23 | Example 24 | Example 25 | Example 26 | Example 27 |
| (A) Polymerizable | Polymerizable compound 1 | | | | | | |
| compound | Polymerizable compound 2 | 20.8 | 20.8 | 20.2 | 19.8 | 19.6 | 18.9 |
| | Polymerizable compound 3 | | | | | | |
| | Polymerizable compound 4 | 24.0 | 24.0 | 23.2 | 22.8 | 22.5 | 21.7 |
| | Polymerizable compound 5 | 21.8 | 21.8 | 21.2 | 20.8 | 20.6 | 19.8 |
| (B) Urethane | Urethane polymer particles 1 | | | | | | |
| polymer | Urethane polymer particles 2 | 27.1 | 27.1 | 26.3 | 25.7 | 25.5 | 24.5 |
| particles | Urethane polymer particles 3 | 6.3 | 6.2 | 6.1 | 5.9 | 5.9 | 5.7 |
| | Urethane polymer particles 4 | | | | | | |
| | Urethane polymer particles 5 | | | | | | |
| (B)′ Acrylic | Acrylic polymer particles 1 | | | | | | |
| polymer | Acrylic polymer particles 2 | | | | | | |
| particles | Acrylic polymer particles 3 | | | | | | |
| | Acrylic polymer particles 4 | | | | | | |
| (C) Polymerization | Polymerization initiator 1 | | 0.1 | 3.0 | | 2.9 | 4.7 |
| initiator | Polymerization initiator 2 | | | | 5.0 | 3.0 | 4.7 |
| | Total (100 mass %) | 100.0 | 100.0 | 100.0 | 100.0 | 100.0 | 100.0 |

| | | | | | | |
|---|---|---|---|---|---|---|
| Test Example 1: Evaluation of matte texture | Unmeasurable | 0 | 0 | 0 | 0 | 0 |
| Test Example 2: Viscosity measurement (mPa · s) | 30300 | 30500 | 31000 | 28200 | 29000 | 29500 |
| Test Example 3: Measurement of elongation at break (%) | Unmeasurable | 41.2 | 42.2 | 40.8 | 39.1 | 38.5 |
| Test Example 4: Tactile sensation test | Unmeasurable | A | A | A | A | A |

As is apparent from Tables 3 to 6, the artificial nail compositions according to the examples were all found to have a matte texture, high flexibility, luxuriousness, and good operability. On the other hand, the artificial nail compositions of the comparative examples could not achieve both a matte texture and high flexibility.

According to the present invention, it is possible to provide an artificial nail composition having a matte texture with reduced gloss and having high flexibility.

What is claimed is:

1. An artificial nail composition comprising:
a component (A): a polymerizable compound;
a component (B): urethane polymer particles; and
a component (C): a polymerization initiator.

2. The artificial nail composition according to claim 1, wherein the urethane polymer particles as the component (B) have an average particle size of 0.8 to 52 μm.

3. The artificial nail composition according to claim 1, wherein a content of the urethane polymer particles as the component (B) is 5 to 50 mass % with respect to a total amount of the artificial nail composition.

4. The artificial nail composition according to claim 1, wherein the urethane polymer particles as the component (B) have a crosslinked structure.

5. The artificial nail composition according to claim 1, wherein the polymerizable compound as the component (A) is at least one selected from the group consisting of a urethane (meth)acrylate oligomer and a polymerizable monomer having an ethylenically unsaturated group.

6. The artificial nail composition according to claim 1, wherein the polymerization initiator as the component (C) is a photopolymerization initiator.

7. The artificial nail composition according to claim 1 comprising:
a component (A): 49 to 94 mass % of a polymerizable compound;
a component (B): 5 to 50 mass % of urethane polymer particles; and
a component (C): 0.1 to 10 mass % of a polymerization initiator.

8. The artificial nail composition according to claim 1, wherein the artificial nail composition is a nail polish, a gel nail, or an acrylic nail.

9. An artificial nail obtained from the artificial nail composition according to claim 1.

10. A method for producing an artificial nail, the method comprising the steps of applying an artificial nail composition containing a polymerizable compound as a component (A), urethane polymer particles as a component (B), and a polymerization initiator as a component (C) onto a nail or a substrate and curing the artificial nail composition.
